(19) United States
(12) Patent Application Publication
(10) Pub. No.: US 2023/0221271 A1
(43) Pub. Date: Jul. 13, 2023

(54) Catalytic Combustion Type Hydrogen Sensor and Method for Manufacturing Same

(71) Applicants: Hyundai Motor Company, Seoul (KR); Kia Corporation, Seoul (KR)

(72) Inventors: Dae Sung Kwon, Seoul (KR); Il Seon Yoo, Suwon-si (KR); Jang Hyeon Lee, Gunpo-si (KR); Dong Gu Kim, Suwon-si (KR); Hyun Soo Kim, Yongin-si (KR)

(21) Appl. No.: 17/880,028

(22) Filed: Aug. 3, 2022

(30) Foreign Application Priority Data
Jan. 11, 2022 (KR) 10-2022-0004232

(51) Int. Cl. G01N 25/22; G01N 33/00

(52) U.S. Cl. CPC G01N 25/22; G01N 33/005

(57) ABSTRACT

An embodiment catalytic combustion type hydrogen sensor includes a protective thin film disposed on an upper surface of a silicon substrate, the protective thin film including an oxide film and a nitride film sequentially laminated, a heater coupled to an upper surface of the nitride film, an anti-icing film disposed on an upper surface of the protective thin film and covering the heater, the anti-icing film including micro-protrusions disposed on an outer surface thereof, and a catalyst layer deposited on an upper surface of the anti-icing film and coated along surfaces of the micro-protrusions of the anti-icing film, wherein the catalyst layer is configured to be heated by the heater to perform a hydrogen reaction for oxidizing hydrogen and to coat the surfaces of the micro-protrusions to prevent water generated through the hydrogen reaction from freezing.

### CROSS-REFERENCE TO RELATED APPLICATIONS

This application claims the benefit of Korean Patent Application No. 10-2022-0004232, filed on Jan. 11, 2022, which application is hereby incorporated herein by reference.

### TECHNICAL FIELD

The disclosure relates to a catalytic combustion type hydrogen sensor and a method for manufacturing the same.

### BACKGROUND

There have recently been increasing interests in hydrogen energy which is eco-friendly alternative energy, which is recyclable, and which causes no environmental contamination problem. In particularly, there has been extensive research for using hydrogen (clean fuel) as the energy source in various fields (for example, fuel cells, internal combustion engines) in line with the trends to develop eco-friendly cars.

However, hydrogen is highly diffusible and thus is likely to leak. If the concentration of hydrogen leaked into the atmosphere is 4% or higher, it easily explodes. Therefore, there is a problem in that, unless safety is guaranteed during use, it is difficult to widely apply hydrogen energy in daily life.

Therefore, there has been ongoing development regarding hydrogen gas detection sensors (hereinafter, referred to as “hydrogen sensors”) capable of early detecting leak of hydrogen gas during actual use, in tandem with research regarding hydrogen energy utilization.

Specifically, various hydrogen sensors are used near hydrogen depositories and hydrogen-related devices to provide safety measures, and it has been necessary to develop highly-reliable hydrogen sensors against hydrogen leak for the sake of car driving and passenger protection.

Hydrogen sensors are generally classified, according to the detection scheme, a semiconductor type, a catalytic combustion type, a field effect transistor (FET) type, an electrolyte type (electrochemical type), an optical fiber type, and a heat conduction type. Catalytic combustion type hydrogen sensors, among the same, have heaters and thus are stable against external environments (for example, change in external temperature and humidity), and they use platinum-group catalysts that are highly hydrogen selective, and thus have the benefits of selectivity and stability.

A general catalytic combustion type hydrogen sensor has a metal wire coil made of platinum or the like and formed in an oxidation catalyst. If combustible gas (for example, hydrogen gas) contacts the surface of the oxidation catalyst after the metal wire (sensing portion) is heated to a specific temperature by applying an electric current thereto, oxidation of the combustible gas causes catalytic combustion and results in reaction heat. This increases the electric resistance of the metal wire, and the electric resistance of the metal wire changed in this manner is converted into an electric signal, thereby sensing combustible gas.

If combustible gas contacts oxidation catalyst and is fully oxidated, water (H₂O) is generated as a byproduct of the hydrogen reaction, and the generated water, if exposed to a low-temperature environment, causes icing on the sensor surface. Such icing blocks contact of combustible gas and causes the problem of degraded sensitivity and performance of the sensor.

There is another problem in that a large amount of power is needed to make a high-temperature environment for the sensing portion, and constant driving based on a battery is thus impossible, and this makes it difficult to detect hydrogen leak while a hydrogen car or the like is powered off. An additional heater may be applied to solve the above-mentioned problem of low-temperature icing, but this additionally increases power consumption and causes the same problem.

Meanwhile, a catalyst may be applied in a dispensing type to increase the surface area of the catalyst as a conventional method for improving the performance of a hydrogen sensor, but this poses a problem in that the catalyst is not applied uniformly due to large errors in connection with the amount of applied catalyst and the position in which the same is applied.

The above descriptions regarding background technologies have been made only to help understanding of the background of the disclosure, and are not to be deemed by those skilled in the art to correspond to already-known prior arts.

### SUMMARY

The disclosure relates to a catalytic combustion type hydrogen sensor and a method for manufacturing the same. Particular embodiments relate to a catalytic combustion type hydrogen sensor and a method for manufacturing the same, wherein water generated as a result of a hydrogen reaction is prevented from freezing, and a uniform catalyst layer is formed such that the catalyst layer has an increased surface area, thereby providing an anti-icing film which improves the sensitivity and performance of a sensor.

Embodiments of the disclosure can solve problems in the art, and an embodiment of the disclosure provides a catalytic combustion type hydrogen sensor and a method for manufacturing the same, wherein a protective thin film, a heater, and a catalyst layer are laminated on a silicon substrate, an anti-icing film is formed between the catalyst layer and the protective thin film such that water generated as a result of a hydrogen reaction is prevented from freezing, and a uniform catalyst layer is formed such that the catalyst layer applied to the upper end of the anti-icing film has an increased surface area, thereby improving the sensitivity and performance of a sensor.

In accordance with an embodiment of the disclosure, a catalytic combustion type hydrogen sensor includes a silicon substrate, a protective thin film which is formed on the upper surface of the silicon substrate and has an oxide film and a nitride film sequentially laminated, a heater which is coupled to the upper end of the nitride film and configured to receive power applied from the outside so as to perform a heating function, an anti-icing film which is formed on the upper surface of the protective thin film to cover and thus insulate the heater and has micro-protrusions formed on the outer surface thereof to prevent freezing of generated water, and a catalyst layer deposited on the upper surface of the anti-icing film, heated by the heater to perform a hydrogen reaction for oxidizing hydrogen, and coated along the surfaces of the micro-protrusions of the anti-icing film such that the micro-protrusions prevent water generated through the hydrogen reaction from freezing.

The anti-icing film of the catalytic combustion type hydrogen sensor according to embodiments of the disclosure may include a first thin film configured to cover the heater and a second thin film formed on the upper surface of the first thin film to insulate the heater, and the micro-protrusions may be formed on the outer surface of the second thin film.

In connection with the catalytic combustion type hydrogen sensor according to embodiments of the disclosure, the micro-protrusions of the anti-icing film may be grown through glancing angle deposition (GLAD).

The heater of the catalytic combustion type hydrogen sensor according to embodiments of the disclosure may include a connection part electrically connected to the outside and a heating part configured to heat the catalyst layer when power is applied through the connection part.

The anti-icing film of the catalytic combustion type hydrogen sensor according to embodiments of the disclosure may have an opening part formed therethrough such that the upper part of the heater is exposed through the opening part, and may further include a metal pad formed to cover the exposed upper part of the heater and provided on the bottom surface of the opening part to receive power supplied from the outside.

The catalyst layer of the catalytic combustion type hydrogen sensor according to embodiments of the disclosure may be coated only in an area identical to an area of the entire upper surface of the anti-icing film, in which the heater embedded inside the anti-icing film is disposed.

The catalytic combustion type hydrogen sensor according to embodiments of the disclosure may further include a through-hole vertically penetrating from the silicon substrate to the lower surface of the oxide film.

A method for manufacturing a catalytic combustion type hydrogen sensor according to embodiments of the disclosure may include forming, on a silicon substrate, a protective thin film having an oxide film and a nitride film sequentially laminated, depositing, on the upper end of the nitride film, a heater configured to perform a heating function by receiving power applied from the outside and then patterning the heater, forming an anti-icing film on the upper part of the heater, forming, on the outer surface of the anti-icing film, surfaces of micro-protrusions configured to prevent freezing of generated water, and coating, on the upper surface of the anti-icing film, a catalyst layer along the surfaces of the micro-protrusions.

In connection with the method for manufacturing a catalytic combustion type hydrogen sensor according to embodiments of the disclosure, the forming the anti-icing film on the upper part of the heater may include annealing the heater after forming a first thin film.

The method for manufacturing a catalytic combustion type hydrogen sensor according to embodiments of the disclosure may further include forming, through the first thin film, an opening part configured to expose the upper part of the heater after annealing the heater, and forming a metal pad formed to cover the exposed upper part of the heater, provided on the bottom surface of the opening part, and connected to a connection part of the heater.

The method for manufacturing a catalytic combustion type hydrogen sensor according to embodiments of the disclosure may further include, after the forming, on the outer surface of the anti-icing film, the surfaces of the micro-protrusions configured to prevent freezing of generated water, etching the anti-icing film such that a metal pad area is exposed.

The method for manufacturing a catalytic combustion type hydrogen sensor according to embodiments of the disclosure may further include, after the coating the catalyst layer on the upper surface of the anti-icing film along the surfaces of the micro-protrusions, polishing the silicon substrate and forming a through-hole.

A catalytic combustion type hydrogen sensor and a method for manufacturing the same, according to embodiments of the disclosure, are advantageous in that a protective thin film, a heater, and a catalyst layer are laminated on a silicon substrate, an anti-icing film is formed between the catalyst layer and the protective thin film such that water generated as a result of a hydrogen reaction is prevented from freezing, and a uniform catalyst layer is formed such that the catalyst layer applied to the upper end of the anti-icing film has an increased surface area, thereby improving the sensitivity and performance of a sensor.

### BRIEF DESCRIPTION OF THE DRAWINGS

FIG. 1 is a view illustrating a structure of a catalytic combustion type hydrogen sensor according to an embodiment of the present disclosure;

FIG. 2 is a view illustrating a surface structure of a conventional catalytic combustion type hydrogen sensor;

FIG. 3A to FIG. 5C are views illustrating a method for manufacturing a catalytic combustion type hydrogen sensor according to an embodiment of the present disclosure; and

FIG. 6 is a flowchart of a method for manufacturing a catalytic combustion type hydrogen sensor according to an embodiment of the present disclosure.

### DETAILED DESCRIPTION OF ILLUSTRATIVE EMBODIMENTS

Throughout the present disclosure, when any part “includes” an element, unless explicitly described to the contrary, it may mean that the part may further include other elements rather than the exclusion of the other elements.

In addition, the terms such as a first and/or a second may be used to describe various elements, but the terms may be merely used to distinguish the element from other elements. For example, although not beyond the scope of rights according to the concept of the present disclosure, the first element may be referred to as a second element, and similarly, the second element also may be referred to as a first element.

Hereinafter, the configuration and the operating principle of various embodiments of the disclosed disclosure will be described in detail with reference to the accompanying drawings.

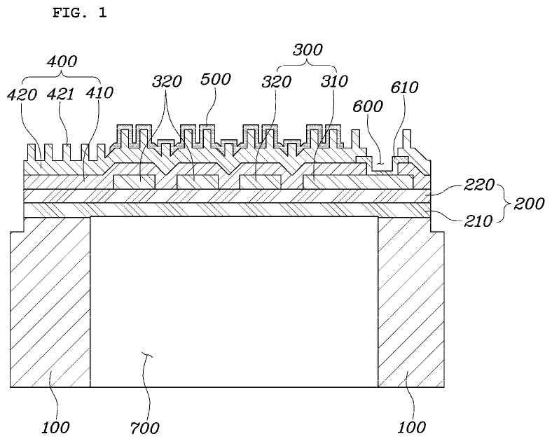

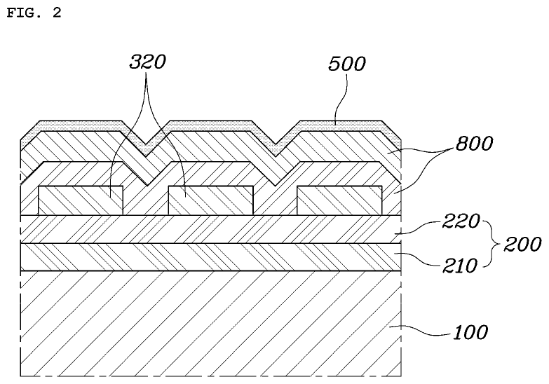

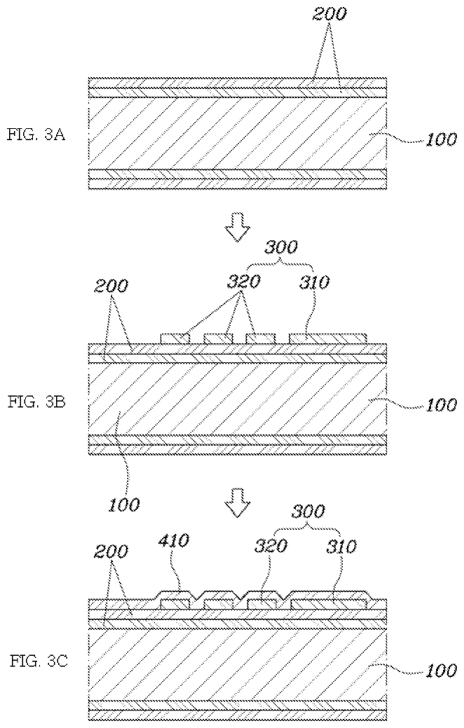

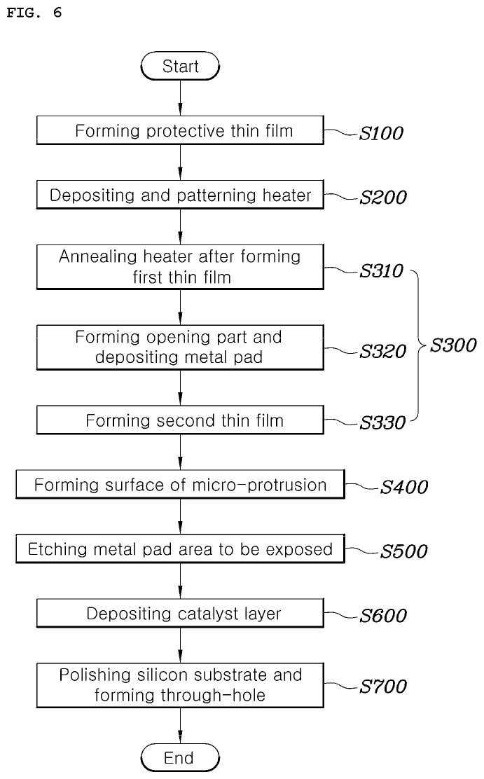

FIG. 1 is a view illustrating a structure of a catalytic combustion type hydrogen sensor according to an embodiment of the present disclosure, FIG. 2 is a view illustrating a surface structure of a conventional catalytic combustion type hydrogen sensor, FIG. 3A to FIG. 5C are views illustrating a method for manufacturing a catalytic combustion type hydrogen sensor according to an embodiment of the present disclosure, and FIG. 6 is a flowchart of a catalytic combustion type hydrogen sensor according to an embodiment of the present disclosure.

In order to help the understanding of embodiments of the present disclosure, the chronic problems of a general catalytic combustion type hydrogen sensor are first described through the structure or the operating principle thereof, and then the key features of elements of embodiments of the present disclosure for solving the problems will be described together.

A general catalytic combustion type hydrogen sensor may have a structure in which a metallic wire coil such as platinum is formed in an oxidation catalyst, and may have, as a basic driving condition, a state where the oxidation catalyst is heated to an appropriate temperature (is generally required to be a high temperature at the level of 70-80° C.) by making current flow through a metallic wire (a detection part). In addition, in order to heat the oxidation catalyst, it may be common that a separate heater 300 (see FIG. 1) is embedded therein.

Therefore, in order to drive a hydrogen sensor, it may be necessary to foster a high temperature environment for an oxidation catalyst, and thus high consumption power may be generally required. That is, in the development of a hydrogen sensor, even if the performance of a hydrogen sensor could be improved, it may be preferable to avoid a structure in which consumption power is increased compared to the existing sensor.

Describing the operating principle of a hydrogen sensor, when a combustible gas (e.g., a hydrogen gas) comes into contact with the surface of an oxidation catalyst under the above driving condition, the combustible gas may be oxidized so that a catalytic combustion occurs, thereby generating reaction heat. Accordingly, the electrical resistance of a metallic wire may increase, and thus the combustible gas may be detected through detecting an electrical signal into which the changeable electrical resistance of the metallic wire is converted.

When a combustible gas is completely oxidized due to coming into contact with an oxidation catalyst, water may be generated as a by-product of a hydrogen reaction, and when generated water is exposed to a low-temperature environment, a freezing phenomenon may occur on the surface of a sensor. The freezing phenomenon may block the combustible gas to be in contact with an oxidation catalyst so that sensitivity and performance of the sensor are degraded. In order to solve the low-temperature freezing problem, an additional heater 300 may be applied thereto. However, it may be undesirable in that consumption power increases.

Therefore, the catalytic combustion type hydrogen sensor according to embodiments of the present disclosure may have an anti-icing film 400 formed thereon, which has a surface of a special shape configured to prevent generated water from remaining on the surface of a sensor, may have a catalyst layer 500 which is applied to the upper end surface of the anti-icing film 400, and thus may prevent water generated in the hydrogen reaction from freezing without applying an additional heater 300.

On the other hand, in order to improve performance of a hydrogen sensor, there may be a method of increasing the surface area of the catalyst layer 500 in addition to a method of preventing freezing of generated water. That is, as a reaction area thereof increases, sensitivity and performance of a sensor may be improved. To this end, in a case of a conventional sensor, a catalyst may be applied using a “dispensing method”.

Here, the dispensing method is a method of dispersing catalyst particles and a structure capable of increasing the surface area thereof and catalyst particles into a solvent, and injecting the dispersion solution formed thereby onto a portion required of catalyst application, so that the catalyst is applied onto the portion since only the solvent evaporates.

In the case of the dispensing method, an injection needle should be positioned at the upper end of the heater 300, and only a predetermined amount of the dispersion solution should be injected thereinto. Therefore, there may be a problem in that controlling the position and the amount are difficult. Therefore, the concentration of catalyst particles dispersed into the dispersion solution may be non-uniform, and thus non-uniformity in performance of a sensor may be caused.

Therefore, according to the catalytic combustion type hydrogen sensor according to embodiments of the present disclosure, a special shape capable of increasing the surface area thereof may be provided on the outer surface of the anti-icing film 400, and the catalyst layer 500 may be applied onto the upper end surface of the anti-icing film 400. Therefore, the catalyst layer 500 may have an increased surface area and the catalyst layer 500 may have uniformity, thereby improving sensitivity and performance of the sensor.

Hereinafter, the technical feature of each element of embodiments of the present disclosure will be described in detail with reference to the accompanying drawings.

FIG. 1 is a view illustrating a structure of a catalytic combustion type hydrogen sensor according to an embodiment of the present disclosure, and FIG. 2 is a view illustrating a surface structure of a conventional catalytic combustion type hydrogen sensor.

Referring to FIG. 1, the catalytic combustion type hydrogen sensor according to embodiments of the present disclosure may include a silicon substrate 100, a protective thin film 200 which is formed on the upper surface of the silicon substrate 100 and has an oxide film 210 and a nitride film 220 sequentially laminated, a heater 300 which is coupled to the upper end of the nitride film 220 and configured to receive power applied from the outside so as to perform a heating function, an anti-icing film 400 which is formed on the upper surface of the protective thin film 200 to cover and thus insulate the heater 300 and has micro-protrusions 421 formed on the outer surface thereof to prevent freezing of generated water, and a catalyst layer 500 deposited on the upper surface of the anti-icing film 400, heated by the heater 300 to perform a hydrogen reaction for oxidizing hydrogen, and coated along the surfaces of the micro-protrusions 421 of the anti-icing film 400 such that the micro-protrusions 421 prevent water generated through the hydrogen reaction from freezing.

Specifically, as illustrated in FIG. 1, the catalytic combustion type hydrogen sensor according to embodiments of the present disclosure may be provided with the silicon substrate 100 and may have the protective thin film 200 which has the oxide film 210 formed to cover the upper part of the silicon substrate 100 and the nitride film 220 formed to cover the upper portion of the oxide film 210, so as to protect the silicon substrate 100. Here, the oxide film 210 may be made of SiO₂, and the nitride film 220 may be made of Si₃N₄.

The heater 300, which is configured to receive power applied from the outside so as to perform a heating function, may be coupled to and disposed on the upper end of the nitride film 220 formed on the upper side of the protective thin film 200. The heater 300 may function to heat the catalyst layer 500 to a predetermined temperature in order for the driving of the catalytic combustion type hydrogen sensor, and may be made of a metal material such as molybdenum (Mo) having a high melting point and good thermal conductivity.

The silicon substrate 100 and the protective thin film 200 formed in the above manner also may be the same as those of the conventional catalytic combustion type hydrogen sensor illustrated in FIG. 2. Here, referring to FIG. 2, an insulation thin film 800, which is configured to cover the heater 300 so as to perform insulation thereof, may be formed on the upper surface of the protective thin film 200. The insulation thin film 800 may include SiO₂ which is an insulation body and a separate thin film (for example, a nitride thin film made of Si₃N₄) configured to protect the insulation body.

In addition, the catalyst layer 500, which is heated by a heating pall 320 of the heater 300 and reacts with hydrogen, may be deposited on the upper surface of the insulation thin film 800. For reference, there is a problem in that generally, hydrogen has a low reactivity with a metal material or a semiconductor material. Therefore, in order for the detection performance improvement of a hydrogen sensor, it may be required to form the catalyst layer 500 coated with a catalytic material for improving reactivity with hydrogen. As the catalytic material, a noble metal catalyst such as platinum (Pt) or palladium (Pd) may be mainly used. In particular, in the case where palladium selectively adsorbs hydrogen, the palladium may undergo changes in mass, volume, electrical resistance, an optical constant, and the like. Therefore, palladium may be used as a hydrogen sensor by measuring the changes.

In other words, the catalyst layer 500 may be made of a noble metal catalyst such as platinum or palladium. However, the above description may be merely an exemplary description for helping the understanding of the present disclosure, and the contents of the present disclosure may not be limited by the above description.

Referring again to FIG. 1, the catalytic combustion type hydrogen sensor according to embodiments of the present disclosure may have the anti-icing film formed on the upper surface of the protective thin film 200 and configured to cover and thus insulate the heater 300. The feature of the anti-icing film may be the same as that of the insulation thin film 800 of FIG. 2. However, it may be known that there is a difference in that the anti-icing film has the micro-protrusions 421 formed on the outer surface thereof to prevent freezing of generated water. That is, according to embodiments of the present disclosure, the anti-icing film 400, which functions as the conventional insulation thin film 800 and can prevent freezing on the surface thereof, may be formed thereon.

In addition, the catalyst layer 500 may be coated and deposited on the upper surface of the anti-icing film 400 along the surfaces of the micro-protrusions 421. Therefore, the catalyst layer 500 may naturally form the surfaces on which the micro-protrusions 421 are formed, and water generated after reaction with hydrogen may be prevented from freezing by the surfaces formed thereby.

Furthermore, the micro-protrusions 421 may be formed in a shape in which multiple pillars are spaced apart with a predetermined distance from each other, and thus there may be an effect that the surface area of the catalyst layer 500 applied to the upper end of the anti-icing film 400 is increased.

For reference, the wording “the shape in which multiple pillars are spaced apart with a predetermined distance from each other” may be merely an exemplary description to help the understanding of the present disclosure, and it should not be interpreted that the shape of the micro-protrusions 421 is limited by the above description.

In addition, the micro-protrusions 421 formed on the outer surface of the anti-icing film 400 will be described in more detail. The micro-protrusions 421 may be configured to form an anti-icing surface, and specifically, it may be understood that the micro-protrusions are intended to form a superhydrophobic surface. Here, “superhydrophobic” may mean a physical property in which the surface of a substrate is not wet with liquid.

That is, when a superhydrophobic surface is formed, water may fall down on the surface thereof and thus may be easily removed. Accordingly, water on the surface of the hydrogen sensor, which remains before ice crystals are formed, may be removed. Therefore, the freezing problem at low-temperatures may be solved. At this time, it also may be natural that the hydrogen sensor is formed to be slightly tilted or to have a predetermined angle such that water remaining on the superhydrophobic surface flows down.

For reference, the method for implementing a superhydrophobic surface may be largely divided into a method of changing the surface shape of a substrate (a first method) and a method of coating a hydrophobic chemical material on the surface of a substrate (a second method). In embodiments of the present disclosure, in order to help the understanding of embodiments of the present disclosure, the method will be described with reference to the first method of forming micro- or nano-patterns on the surface of a substrate, controlling the roughness thereof, and changing the contact angle between a liquid and the surface of a substrate.

However, in the case of the micro-protrusions 421 positioned in an area where the catalyst layer 500 is not applied, the second method may be applied thereto, and a different method may be applied thereto according to a specific environmental change for improving performance of a hydrogen sensor. That is, the first method above presented may be merely an exemplary description for helping the understanding of embodiments of the present disclosure, and the contents of the present disclosure may not be limited by the above description.

In conclusion, the catalytic combustion type hydrogen sensor according to embodiments of the present disclosure may include a superhydrophobic surface having a micro- or nano-structure formed by the micro-protrusions 421 of the anti-icing film 400, and thus can solve the freezing problem on the surface of a sensor at a low-temperature.

On the other hand, the anti-icing film 400 according to the catalytic combustion type hydrogen sensor of embodiments of the present disclosure may include a first thin film 410 configured to cover the heater 300 and a second thin film 420 formed on the upper surface of the first thin film 410 to insulate the heater 300, and the micro-protrusions 421 may be formed on the outer surface of the second thin film 420.

That is, according to the catalytic combustion type hydrogen sensor, the upper part of the heater 300 may be covered by the anti-icing film 400 including the first thin film 410 and the second thin film 420, and the micro-protrusions 421 may be provided on the outer surface of the second thin film 420 to form a superhydrophobic surface. Here, the first thin film 410 may be made of SiO₂ which is an insulation body, and the second thin film 420 may be made of Si₃N₄.

At this time, a material constituting the micro-protrusions 421, through deposition, may be made of Si₃N₄ which is the same as that of the second thin film 420. However, considering the property of the superhydrophobic surface which allows water having high electrical conductivity to be easily removed from the surface thereof, and the feature in which the superhydrophobic surface is formed to cover the upper part of the heater 300 to which power is applied, it may be natural that various other materials having an insulating property are used therefor.

That is, although FIG. 1 illustrates that the micro-protrusions 421 of the anti-icing film 400 are made of the same material as that of the second thin film 420, it is merely to help the understanding of embodiments of the present disclosure, and the contents of the present disclosure may not be limited by the shapes of the drawings.

On the other hand, in the catalytic combustion type hydrogen sensor of embodiments of the present disclosure, the micro-protrusions 421 of the anti-icing film 400 may be grown through a glancing angle deposition (GLAD) method.

The glancing angle deposition method may mean a bottom-up nano-structure formation technique, and may have advantages capable of forming a basic frame of the structure and precisely adjusting the detailed shape of the nanostructure during the deposition process.

The glancing angle deposition method may use a physical vapor deposition (PVD) such as a sputtering method, or a plasma enhanced chemical vapor deposition (PECVD) apparatus and a vacuum deposition apparatus such as an electron beam melting (EBM).

That is, the micro-protrusions 421 according to the embodiments of the present disclosure may be grown and formed through the glancing angle deposition method using the above method or apparatus. At this time, the inclination or the angle of the silicon substrate 100, the rotational speed thereof, and the vapor flow rate of the material constituting the micro-protrusions 421 may be adjusted to precisely form the shape or the density of the micro-protrusions 421.

Therefore, a nano-structure having various shapes may be formed over the entire surface thereof even without adding additional processes such as a separate etching process or a high-temperature synthesis process.

In addition, when deposition is performed in the state where the silicon substrate 100 is inclined during a deposition process, there may occur a problem that the particles deposited first themselves interrupt the progress of particles deposited later. It may be called a “self-shadowing effect”. Therefore, the rear surfaces of particles may be covered due to the effect, and thus an area, on which materials are not deposited, may occur. However, according to embodiments of the present disclosure, materials may be deposited in a shape in which multiple pillars are spaced apart with a predetermined distance from each other. Therefore, as a result, materials may be deposited in the same shape as that of the micro-protrusions 421 of embodiments of the present disclosure.

In addition, the catalyst layer 500 may be deposited on the upper surface of the anti-icing film 400 on which the micro-protrusions 421 are formed, to increase the surface area of the catalyst layer 500 and to uniformly form the catalyst layer 500, thereby ultimately improving sensitivity and performance of the sensor.

On the other hand, the heater 300 of the catalytic combustion type hydrogen sensor according to embodiments of the present disclosure may include a connection part 310 electrically connected to the outside and a heating part 320 configured to heat the catalyst layer 500 when power is applied through the connection part 310.

When power is applied from the outside through the connection part 310, current may flow through the heater 300, and the current may be delivered to the heating part 320 of the heater 300. In addition, the catalyst layer 500 may be deposited on the upper side of the heating part 320 of the heater 300 while having the anti-icing film 400 interposed therebetween.

Therefore, as the temperature of the heating part 320 increases due to the current delivered through the connection part 310, the catalyst layer 500 may be heated and thus activated.

For reference, the anti-icing film 400 not only may allow the heat generated in the heating part 320 to be delivered to the catalyst layer 500, but also may prevent the currents of the catalyst layer 500 and the heating part 320 from interfering with each other by the first thin film 410 and the second thin film 420.

On the other hand, the anti-icing film 400 of the catalytic combustion type hydrogen sensor according to embodiments of the present disclosure may include an opening part 600 formed therethrough, and the upper part of the heater 300 may be exposed through the opening part 600. In addition, the anti-icing film may further include a metal pad 610 formed to cover the exposed upper part of the heater 300 and provided on the bottom surface side of the opening part 600 to receive power supplied from the outside.

The metal pad 610 may function as a kind of electrode for connecting an external power supply and the heater 300. That is, the metal pad 610 may correspond to a configuration for applying power from the outside to the heater 300. Therefore, it may be preferable to understand that the wording “the upper part of the heater 300” means the upper part of the connection part 310 of the heater 300. However, the heater 300 may be variously designed to improve temperature uniformity of the hydrogen sensor. Therefore, it may be natural that the heater is also designed differently from the present embodiment according to the design change thereof.

That is, in the following description, although it will be described as an embodiment that the metal pad 610 is connected to the upper part of the connection part 310 of the heater 300, it may be merely for helping the understanding of embodiments of the present disclosure, and the contents of the present disclosure may not be limited by the description.

When described in more detail with reference to FIG. 1, the opening part 600, which is formed to penetrate up to the connection part 310 of the heater 300 such that the upper part of the connection part 310 of the heater 300 is exposed therethrough, may be formed through the anti-icing film 400, and the metal pad 610 may be provided on the bottom surface side of the opening part 600. The lower end part of the metal pad 610 may be in contact with the upper part of the heater 300 and thus may cover the exposed upper part of the heater 300. In addition, the upper end part of the metal pad 610 may be connected to an external power supply through the opening part 600.

As a result, an external power supply and the heater 300 may be easily connected through the metal pad 6i0, and thus current may be stably delivered to the heater 300.

On the other hand, the catalyst layer 500 of the catalytic combustion type hydrogen sensor according to embodiments of the present disclosure, among the entire upper surface of the anti-icing film 400, may be coated on only an area which is identical to the area in which the heater 300 embedded inside the anti-icing film 400 is disposed.

That is, the catalyst layer 500 may be formed to be deposited in only a partial area rather than the entire area of the upper surface of the anti-icing film 400. The reason is that in general, the catalyst layer 500 deposited in the form of a thin film may generate stress in the deposition area during the deposition process, and the catalyst layer 500 or a thin film (in the case of embodiments of the present disclosure, it means the anti-icing film 400) onto which the catalyst layer 500 is applied, may be deformed due to the generated stress. Therefore, in order to minimize the influence of the stress, the catalyst may be formed to have as small an area as possible in consideration of the area in which the heater 300 is formed, among the entire area of the anti-icing film 400.

In addition, the catalyst layer 500 may be activated only when heated above a predetermined temperature, and thus may have improved reactivity with hydrogen. Therefore, according to the catalytic combustion type hydrogen sensor of embodiments of the present disclosure, the catalyst layer 500 may be deposited in an area identical to the area in which the heater 300 is disposed, thereby more effectively heating and thus sufficiently activating the catalyst layer 500.

Furthermore, it may be preferable that the catalyst layer 500 is deposited in an area identical to the area in which the heating part 320 of the heater 300 is disposed, among the area in which the heater 300 is disposed. The deposition process of the catalyst layer 500 may be performed in the state where the upper end part of the metal pad 610 connecting an external power supply and the heater 300 is exposed to the outside. Therefore, if a catalyst is deposited on the exposed metal pad 610, a problem may occur in that conduction sensitivity thereof is degraded. Therefore, in order to prevent the conduction problem, the catalyst layer 500 may be disposed at the upper end part of an area identical to the area in which the heating part 320 of the heater 300 is disposed other than the connection part 310 of the heater 300, in which the metal pad 610 is disposed.

Meanwhile, the catalytic combustion type hydrogen sensor according to embodiments of the present disclosure may further include a through-hole 700 formed to vertically penetrate from the silicon substrate 100 to the lower surface of the oxide film 210.

As illustrated in FIG. 1, the through-hole 700, which is formed to penetrate in the vertical direction, may be formed through the silicon substrate 100 to be positioned below the heating part 320 of the heater 300. The through-hole 700 may be formed to penetrate from the silicon substrate 100 to the lower surface of the oxide film 210.

The heat generated in the heater 300 may be discharged to the lower side of the hydrogen sensor through the through-hole 700, to minimize the damage to the silicon substrate 100. Heat required for heating the catalyst layer 500 may be transferred only to the upper part to activate the catalyst layer 500.

FIG. 3A to FIG. 5C are views illustrating a method for manufacturing a catalytic combustion type hydrogen sensor according to an embodiment of the present disclosure, and FIG. 6 is a flowchart of a method for manufacturing a catalytic combustion type hydrogen sensor according to an embodiment of the present disclosure.

Referring to FIG. 3A to FIG. 6, a method for manufacturing the catalytic combustion type hydrogen sensor according to embodiments of the present disclosure may include a step S100 of forming a protective thin film having an oxide film and a nitride film sequentially laminated on a silicon substrate, a step S200 of depositing, on the upper end of the nitride film, a heater for performing a heating function and then patterning the same by receiving power applied from the outside, a step S300 of forming an anti-icing film on the upper part of the heater, a step S400 of forming micro-protrusions on the outer surface of the anti-icing film to prevent freezing of generated water, and a step S600 of coating, on the upper surface of the anti-icing film, a catalyst layer along the surfaces of the micro-protrusions.

Specifically describing with reference to FIG. 3A to FIG. 5C, FIG. 3A illustrates the forming, on the upper surface of the silicon substrate, the protective thin film 200 having the oxide film and the nitride film sequentially laminated (S100), FIG. 3B illustrates the depositing the heater 300 on the upper end of the nitride film and then patterning same (S200), and FIG. 3C illustrates the forming of a first thin film 410 of the anti-icing film on the upper part of the heater. After forming the first thin film, a process S310 of annealing the heater may be performed.

That is, in the manufacturing method of the catalytic combustion type hydrogen sensor according to embodiments of the present disclosure, the step S300 of forming the anti-icing film on the upper part of the heater may include the annealing of the heater after forming the first thin film (S310).

Here, the annealing may mean a heat treatment method of heating a metal material to a predetermined temperature and then slowly cooling the same in order to even the inner crystalline structure of the metal material, remove the internal residual stress so as to reduce hardness and strength of the metal, and improve formability.

Through the annealing, the performance of the heater 300 can be improved, and the internal residual stress of the anti-icing film 400 for performing an insulating function can be reduced.

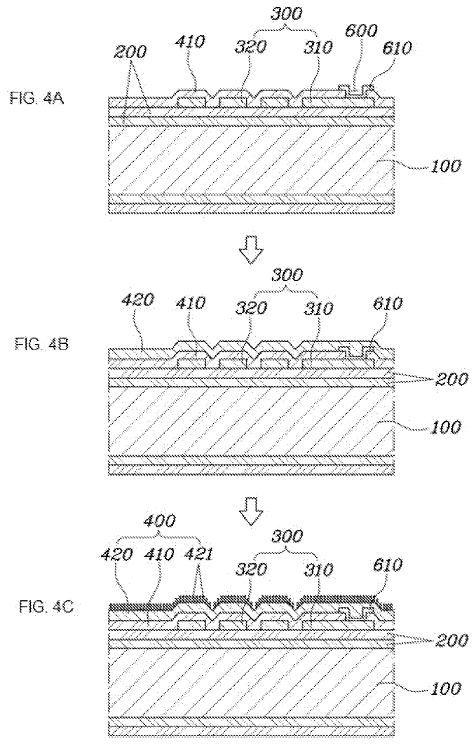

Continually describing, FIG. 4A illustrates the forming of an opening part through the first thin film after annealing and forming a metal pad on the bottom surface side of the opening part (S320). That is, the manufacturing method of the catalytic combustion type hydrogen sensor according to embodiments of the present disclosure may further include the step S320 of forming, through the first thin film, the opening part configured to expose the upper part of the heater therethrough, and forming a metal pad formed to cover the exposed upper part of the heater and provided on the bottom surface side of the opening part to be connected to the connection part of the heater.

The metal pad 610 may be formed to be connected to the connection part 310 of the heater 300, and thus an external power supply and the heater 300 may be easily connected to each other.

In addition, FIG. 4B illustrates depositing a second thin film 420 of the anti-icing film 400 thereon (S330), and FIG. 4C illustrates forming the surfaces of micro-protrusions on the outer surface of the anti-icing film configured to prevent freezing of generated water (S400). In order to form the surfaces of the micro-protrusions on the outer surface of the anti-icing film, the glancing angle deposition method above described may be utilized.

On the other hand, in the process of depositing the second thin film 420 of the anti-icing film 400 and forming the surfaces of the micro-protrusions 421, the metal pad 610 may be covered by the second thin film 420, and thus the previously formed opening part 600 may temporarily disappear. Therefore, the manufacturing method of the catalytic combustion type hydrogen sensor according to embodiments of the present disclosure may further include a step S500 of etching the anti-icing film such that the area corresponding to the metal pad is exposed after the step S400 of forming, on the outer surface of the anti-icing film, the surfaces of the micro-protrusions configured to prevent freezing of generated water.

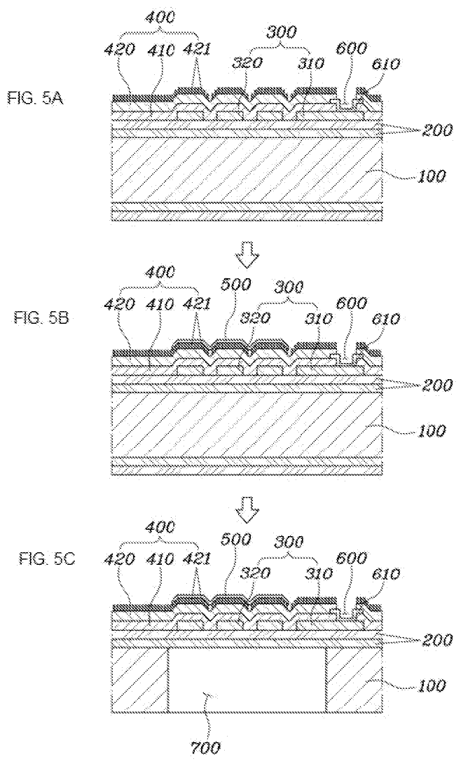

That is, as illustrated in FIG. 5A, the anti-icing film may be etched to allow the metal pad 610 in contact with the connection part 310 of the heater 300 to be exposed to the outside. Therefore, in the catalytic combustion type hydrogen sensor according to embodiments of the present disclosure, although the opening part 600 temporarily disappears in the process of depositing the second thin film 420 of the anti-icing film 400, the opening part may be reliably implemented.

After that, as illustrated in FIG. 5B, a catalyst layer may be deposited and coated on the upper surface of the anti-icing film along the surfaces of the micro-protrusions (S600). A catalyst may be applied to the area of the metal pad 610, which is exposed to the outside, in the deposition process of the catalyst layer 500. Therefore, it may cause a problem in that the conduction sensitivity of the metal pad 610 is degraded. However, as described above, in order to completely solve the problem, the catalyst layer 500 may be deposited only in an area identical to the area in which the heating part 320 of the heater 300 is disposed among the area where the heater 300 is disposed.

The catalyst layer 500 may be deposited along the surfaces of the micro-protrusions 421 so that the catalyst layer 500 having a superhydrophobic surface is formed. Accordingly, water generated by the oxidation reaction of the catalyst layer 500 and hydrogen can be easily removed, and thus the low-temperature freezing problem, which is a chronic problem in a catalytic combustion type hydrogen sensor, can be solved.

In addition, as illustrated in FIG. 5C, the manufacturing method of the catalytic combustion type hydrogen sensor according to embodiments of the present disclosure may further include a step S700 of polishing the silicon substrate and forming a through-hole after the step S600 of coating the catalyst layer on the upper surface of the anti-icing film along the surfaces of the micro-protrusions.

That is, the through-hole 700, which penetrates from the silicon substrate 100 to the lower surface of the oxide film 210, may be formed through the silicon substrate 100, and the through-hole 700 may be formed to be positioned below the heating part 320 of the heater 300. Therefore, heat generated from the heater 300 may be discharged to the lower side of the hydrogen sensor to minimize damage of the silicon substrate 100.

As described above, according to the catalytic combustion type hydrogen sensor and the manufacturing method of same according to embodiments of the present disclosure, the protective thin film 200, the heater 300, and the catalyst layer 500 may be laminated on the silicon substrate 100, and the anti-icing film 400 may be formed between the catalyst layer and the protective thin film 200. Therefore, water generated in the hydrogen reaction can be prevented from freezing. In addition, the surface area of the catalyst layer 500 applied on the upper end of the anti-icing film 400 can be increased and the catalyst layer 500 having uniformity can be formed, thereby improving sensitivity and performance of the sensor.

Although illustrated and described in connection with a specific embodiment of the present disclosure, it should be obvious to a person skilled in the art that the present disclosure may be variously changed and modified without departing from the technical idea of the present disclosure, which is provided by the following claims.

What is claimed is:

1. A catalytic combustion type hydrogen sensor comprising:
a protective thin film disposed on an upper surface of a silicon substrate, the protective thin film comprising an oxide film and a nitride film sequentially laminated;
a heater coupled to an upper surface of the nitride film, the heater being configured to receive power applied from outside;
an anti-icing film disposed on an upper surface of the protective thin film and covering the heater, the anti-icing film comprising micro-protrusions disposed on an outer surface thereof, the micro-protrusions being configured to prevent freezing of generated water; and
a catalyst layer deposited on an upper surface of the anti-icing film and coated along surfaces of the micro-protrusions of the anti-icing film, wherein the catalyst layer is configured to be heated by the heater to perform a hydrogen reaction for oxidizing hydrogen and to coat the surfaces of the micro-protrusions to prevent water generated through the hydrogen reaction from freezing.

2. The catalytic combustion type hydrogen sensor of claim 1, wherein the anti-icing film comprises:
a first thin film covering the heater; and
a second thin film disposed on an upper surface of the first thin film to insulate the heater.

3. The catalytic combustion type hydrogen sensor of claim 2, wherein the micro-protrusions are disposed on an outer surface of the second thin film.

4. The catalytic combustion type hydrogen sensor of claim 1, wherein the micro-protrusions of the anti-icing film are grown through glancing angle deposition.

5. The catalytic combustion type hydrogen sensor of claim 1, wherein the heater comprises:
a connection part electrically connected to the outside; and
a heating part configured to heat the catalyst layer when the power is applied through the connection part.

6. The catalytic combustion type hydrogen sensor of claim 1 wherein the anti-icing film has an opening part formed therethrough such that an upper part of the heater is exposed through the opening part.

7. The catalytic combustion type hydrogen sensor of claim 6, further comprising a metal pad covering the exposed upper part of the heater and provided on a bottom surface of the opening part to receive the power supplied from the outside.

8. The catalytic combustion type hydrogen sensor of claim 1, wherein the catalyst layer is coated only in an area identical to an area of the entire upper surface of the anti-icing film in which the heater embedded inside the anti-icing film is disposed.

9. The catalytic combustion type hydrogen sensor of claim 1, further comprising a through-hole vertically penetrating from the silicon substrate to a lower surface of the oxide film.

10. A method for manufacturing a catalytic combustion type hydrogen sensor, the method comprising:
forming a protective thin film on a silicon substrate, the protective thin film comprising an oxide film and a nitride film sequentially laminated;
depositing a heater on an upper surface of the nitride film and patterning the heater, wherein the heater performs a heating function by receiving power applied from outside;
forming an anti-icing film on an upper part of the heater;
forming micro-protrusions on an outer surface of the anti-icing film, wherein the micro-protrusions prevent freezing of generated water; and
coating a catalyst layer on the outer surface of the anti-icing film along surfaces of the micro-protrusions.

11. The method of claim 10, wherein forming the anti-icing film on the upper part of the heater comprises annealing the heater after forming a first thin film.

12. The method of claim 11, further comprising:
forming an opening part through the first thin film to expose the upper part of the heater after annealing the heater; and
forming a metal pad covering the exposed upper part of the heater, wherein the metal pad is provided on a bottom surface of the opening part and is connected to a connection part of the heater.

13. The method of claim 12, further comprising, after forming the micro-protrusions, etching the anti-icing film to expose a metal pad area.

14. The method of claim 10, further comprising, after coating the catalyst layer on the upper surface of the anti-icing film along the surfaces of the micro-protrusions, polishing the silicon substrate and forming a through-hole.

15. A catalytic combustion type hydrogen sensor comprising:
a protective thin film disposed on an upper surface of a silicon substrate, the protective thin film comprising an oxide film and a nitride film sequentially laminated;
a heater coupled to an upper surface of the nitride film;
an anti-icing film disposed on an upper surface of the protective thin film and covering the heater, the anti-icing film comprising micro-protrusions disposed on an outer surface thereof; and
a catalyst layer deposited on an upper surface of the anti-icing film and coated along surfaces of the micro-protrusions of the anti-icing film.

16. The catalytic combustion type hydrogen sensor of claim 15, wherein the catalyst layer is configured to be heated by the heater to perform a hydrogen reaction for oxidizing hydrogen and to coat the surfaces of the micro-protrusions to prevent water generated through the hydrogen reaction from freezing.

17. The catalytic combustion type hydrogen sensor of claim 15, wherein the anti-icing film comprises:
a first thin film covering the heater; and
a second thin film disposed on an upper surface of the first thin film to insulate the heater, the micro-protrusions being disposed on an outer surface of the second thin film.

18. The catalytic combustion type hydrogen sensor of claim 15, wherein the heater comprises:
a connection part electrically connected to the outside; and
a heating part configured to heat the catalyst layer when the power is applied through the connection part.

19. The catalytic combustion type hydrogen sensor of claim 15 wherein the anti-icing film has an opening part formed therethrough such that an upper part of the heater is uncovered by the anti-icing film.

20. The catalytic combustion type hydrogen sensor of claim 15, further comprising a through-hole vertically penetrating from the silicon substrate to a lower surface of the oxide film.
